(19) United States
(12) Patent Application Publication
(10) Pub. No.: US 2026/0083664 A1
(43) Pub. Date: Mar. 26, 2026

(54) INTRATHECAL ADMINISTRATION OF T REGULATORY CELLS IN THE TREATMENT OF MULTIPLE SCLEROSIS

(71) Applicant: GDANSKI UNIWERSYTET MEDYCZNY, Gdansk (PL)

(72) Inventors: Piotr TRZONKOWSKI, Gdansk (PL); Kamil CHWOJNICKI, Gdansk (PL)

(21) Appl. No.: 19/407,506

(22) Filed: Dec. 3, 2025

Related U.S. Application Data
(63) Continuation of application No. 17/755,016.

(30) Foreign Application Priority Data
Dec. 12, 2019 (PL) P.432200

(51) Int. Cl. A61K 9/00; A61K 40/11; A61K 40/22; A61K 40/41; A61P 25/00; C12N 5/0783

(52) U.S. Cl. CPC A61K 9/0019; A61K 40/11; A61K 40/22; A61K 40/416; A61P 25/00; C12N 5/0637; A61K 2239/31

(57) ABSTRACT

The invention relates to the medicinal product consisting of CD3+CD4+CD25+CD127− T regulatory cells administered intrathecally in the treatment of multiple sclerosis.

The invention relates the medicinal product consisting of CD3+CD4+CD25+CD127− T regulatory cells for clinical use in the treatment. The product is administered intrathecally in the treatment of multiple sclerosis.

Multiple sclerosis (MS) is an immune-mediated disease in which autoimmune T conventional cells (Tconvs) sensitized against myelin sheath break blood-brain barrier and destroy neurons of the central nervous system (CNS). It is hypothesized that CD4⁺CD25^{high}CD127⁻FoxP3⁺ regulatory T cells (Tregs) may inhibit this destruction through suppressive activity exerted on Tconvs.

Tregs lymphocytes constitute for about 1% of all peripheral blood lymphocytes, but are important for maintaining the tolerance of their own tissues. Lack of regulatory T cells leads to a number of autoimmune diseases and hypersensitivity, as seen in the case of patients with X-linked immunodeficiency syndrome, polyendocrinopathy and enteropathy (IPEX). One of such autoimmune syndromes is also multiple sclerosis.

Treg lymphocytes can be called “intelligent steroids” because as steroids, they inhibit inflammatory reactions and act immunosuppressively, but in contrast the physiological suppressor effect of Treg cells concerns only pathological reactions (eg. directed against its own tissues). The results of clinical trials, including the inventors observations, indicate that therapy with Treg lymphocytes is safe and does not impair the immune response against foreign and dangerous antigens (viruses, bacteria, cancer cells).

The invention provides the way of administration of medicinal product consisting of CD3+CD4+CD25+CD127− T regulatory cells via intrathecal injection.

The subject of invention is the medicinal product consisting of CD3+CD4+CD25+CD127− T regulatory cells.

The product is administered intrathecally for the treatment of patients diagnosed with multiple sclerosis

The product is administered intrathecally.

The product is administered in multiple sclerosis.

### DESCRIPTION OF THE FIGURES

FIG. 1—presents the clinical outcomes in the study

The patients underwent protocol-planned neurological examinations throughout the trial. The quality of life was assessed with EQ-5D questionnaire (EQ-5D) and the physical/neurological status was monitored with the EDSS scale and the components of MSFC scale, such as Timed 25-Foot Walk (FWT), Dominant (9-HPT P) and Non-Dominant (9-HPT L) Nine-Hole Peg Test (9-HPT) and the Paced Auditory Serial Addition Test (PASAT). The scores are presented throughout the follow-up separately for the patients administered intravenously and intrathecally as medians (minimum-maximum) and dots represent raw data.

FIG. 2—presents CONSORT Flow Diagram

FIG. 3—presents the progression of disease in the CNS using MRI

The patients underwent protocol-planned MRI examinations throughout the trial. The most important changes are presented as the index of changes of the total volume of the plaques on FLAIR sequence, of the volume of the five the biggest plaques on FLAIR sequence, and of the number of plaques. The index of changes on ‘y’ axes was calculated from the individual values of the variables from day ‘0’ (immediately before administration of Tregs) which were treated as ‘100’ and the changes in the following examinations were calculated proportionally. The changes in the values of contrast-enhanced lesions and the number of microbleeds are presented as absolute numbers. The indexes and the absolute values are presented throughout the follow-up separately for the patients administered intravenously and intrathecally as medians (minimum-maximum) and dots represent raw data. The between-group differences are linked with the line and marked with asterisk (*) and the changes over time in particular group are marked with the line and hash (#).

FIG. 4—presents additional data on the progression of disease in the CNS using MRI

The patients underwent planned MRI examinations throughout the trial. The charts present changes in the volumes of particular structures in CNS (grey matter, white matter, brain, brain cortex, brain parenchyma, cerebrospinal fluid) and T1 hypointensities in grey and white matter throughout the trial. The data are presented as the index of changes on ‘y’ axes, in which the individual values of the variables from day ‘0’ were treated as ‘100’ and the changes in the following examinations were calculated proportionally. The indexes are presented throughout the follow-up separately for the patients administered intravenously and intrathecally as medians (minimum-maximum) and dots represent raw data.

FIG. 5—presents the levels of Tregs and Tconvs throughout the study

Flow cytometry analysis, representative flow histograms and gating strategies [A]. The analysis of flow data started from forward vs. side scatter dot-plot and generation of lymph gate (P1). This was used to create CD3 vs. CD4 dot-plot and CD3+CD4+ T cell gate(P2). This gate was used to analyze Treg cells (dot-plots in the left column) and CD4+ Tconv cells (dot-plots in the right column). Treg gate covering CD127-CD25high cells (P3-left column) was established and the expression of FoxP3 in this gate was verified in FoxP3 vs. Helios dot-plot, which was then used to generate three gates: all Tregs FoxP3+(P4 left column), thymic tTregs FoxP3+Helios+ (P5) and peripheral pTregs FoxP3+Helios− (P6). The levels of all Tregs (CD3+CD4+CD25^{high}CD127-FoxP3+), thymic tTregs (CD3+CD4+CD25^{high}CD127-FoxP3+Helios+) and peripheral pTregs (CD3+CD4+CD25^{high}CD127-FoxP3+Helios−) are shown in charts [B]. In addition, CD62L vs. CD45RA dot-plots were generated from TregFoxP3+ gate and TconvFoxP3− gate to assess the percentages of naïve/memory cells. The levels of Tn (CD62L⁺CD45RA⁺-Q2), Tcm (CD62L⁺CD45RA⁻Q1), and Tem (CD62L⁻CD45RA⁻-Q3) within Tregs (CD3⁺CD4⁺CD25^{high}CD127⁻FoxP3⁺) and Tconvs (CD3⁺CD4⁺CD25^{low/-}CD127⁺FoxP3⁻) are shown in charts [C]. The percentage of Tregs FoxP3+ expressing the following markers: CCR10, CXCR4, CCR4, CD103, CCR8, CD18, CD39, CD73, CTLA-4, PD-1, 4-1BB and, OX40 was analysed from all three Treg gates (P4 to P6—left-column histogram in [A]). The example histogram shows the analysis of CD39 expression (Q2-1 quadrant in left-column histogram in [A]) but the same was performed for other markers. The files acquired for Treg analysis were also used to assess the expression of the same markers on Tconv cells. Tconvs were found through inversion of the position of P3 and P4 gates used originally for Treg analysis. Tconv gate covering CD127+CD25low/− cells (P3-right-column histogram in [A]) was established and the lack of FoxP3 in this gate was verified in the dot-plot FoxP3 vs. Helios, which was then used to generate Tconv FoxP3− gate (P4 right-column histogram in [A]). The percentage of Tconvs expressing the following markers: CCR10, CXCR4, CCR4, CD103, CCR8, CD18, CD39, CD73, CTLA-4, PD-1, 4-1BB and, OX40 was then analyzed from right-column histogram P4 gate. The example shows the analysis of CD39 expression (Q2-1 quadrant in right column histogram in [A]). The levels of Tregs and Tconvs (from P4 gate) expressing the markers are presented as the heatmap. The tree of clusters was ordered to find the markers with contrasting expression between Tregs and Tconvs [heatmap D, detailed levels also in FIG. 6]. A similar clustering analysis was performed to compare and contrast thymic tTregs (CD3+CD4+CD25highCD127−FoxP3+Helios+ from P5 in left-column histogram in [A]) and peripheral pTregs (CD3+CD4+CD25highCD127−FoxP3+Helios− from P6 in left-column histogram in [A]) [heatmap E].

The cut off for positive signals in the flow-analysis shown in [A] was established based on isotype controls and fluorescence minus one (FMO) gatings. The arrows show hierarchy of the gating. The number of dots in particular dot-plots was reduced to clearly show the populations. The flow cytometer was operationally qualified (OQ) by independent service operator and regular quality control was performed with CS&T beads (BDBioscience, USA). In charts [B] and [C], the percentages of cells are presented at administration of Tregs preparation (day ‘0’), +3, +6, and +12 months post-administration separately for iv. group and tc. group. The results are presented as medians (minimum-maximum) and dots represent raw data. The asterisks [*] throughout the figure mark significant differences.

FIG. 6—presents the levels of subsets of Tregs and Tconvs throughout the study

(percentage values corresponding to the heatmap in FIG. 5)

A. The percentages of subsets of Tregs and Tconvs expressing chemokines receptors or integrins are presented at administration of Tregs preparation (day ‘0’), +3, +6, and +12 months post-administration separately for each trial group. The percentages of CCR10⁺, CXCR4⁺, CCR4⁺, CD103⁺, CCR8⁺ and CD18⁺ cells within Tregs (CD3⁺CD4⁺CD25^{high}CD127⁻FoxP3⁺) and Tconvs (CD3⁺CD4⁺CD25^{low/-}CD127⁺FoxP3⁻) are shown. The results are presented as medians (minimum-maximum) and dots represent raw data.

B. The percentages of subsets of Tregs and Tconvs expressing receptors and other molecules important for the functioning of Tregs are presented at administration of Tregs preparation (day ‘0’), +3, +6, and +12 months post-administration separately for each trial group. The percentages of CD39⁺, CD73⁺, CTLA-4⁺, PD-1⁺, 4-1BB⁺, and OX40⁺ cells within Tregs (CD3⁺CD4⁺CD25^{high}CD127⁻FoxP3⁺) and Tconvs (CD3⁺CD4⁺CD25^{low/-}CD127⁺FoxP3⁻) are shown. The results are presented as medians (minimum-maximum) and dots represent raw data.

C. The subsets of tTregs (CD3⁺CD4⁺CD25^{high}CD127⁻FoxP3⁺Helios⁺) and pTregs (CD3⁺CD4⁺CD25^{high}CD127⁻FoxP3⁺Helios⁻) are presented at administration of Tregs preparation (day ‘0’), +3, +6, and +12 months post-administration separately for each trial group. Only the subsets that differ significantly are shown: CCR10⁺, CD103⁺, CD39⁺, CD73⁺, and CTLA-4⁺. The results are presented as medians (minimum-maximum) and dots represent raw data.

FIG. 7 presents the levels of serum cytokines throughout the study

The levels of cytokines in serum of the patients treated with intravenous or intrathecal injection of Tregs are presented as the heatmap. The tree of clusters was ordered to find the clusters of cytokines which levels contrast between these two groups of patients (detailed levels also in FIG. 8). The levels of cytokines are presented at administration of Tregs preparation (day ‘0’), +14 days, +3, +6, +9, and +12 months post-administration separately for each trial group. The asterisks [*] mark the cytokines which levels were significantly different between these two groups of patients.

FIG. 8—presents the serum cytokine levels throughout the study

(percentage values corresponding to the heatmap in FIG. 7)

The levels of cytokines in the serum of the patients are presented at administration of Tregs preparation (day ‘0’), +14 days, +3, +6, +9, and +12 months post-administration separately for each trial group. The cytokines which levels differed significantly between groups are marked in bold. These include TGFalpha and related to inflammation MCP3, CXCL8 and IL1RA. The results are presented as medians (minimum-maximum) and dots represent raw data.

The present invention are illustrated by the following examples, which are not its limitation.

### Protocol of the Study

The study was conducted according to the Declaration of Helsinki principles. The protocol has been registered in the EudraCT database under the number 2014-004320-22 and received approval from the Institutional Review Board of the Medical University of Gdansk (no. NKBBN/414/2012 and NKBBN/414-163/2017). Written informed consent was received from all the participants at the recruitment, before any medical procedure was commenced.

Fourteen MS patients (18-55 yo) were recruited into the two groups treated with Tregs either intravenously (iv. n=11) or intrathecally (tc. n=3) (Table 1 and FIG. 2). One patient from iv. group dropped out of the trial due to pregnancy during the follow-up. The inclusion criteria were as follows: relapsing-remitting form of MS (diagnosed according to the McDonalds criteria or revised McDonald criteria) with at least 1 relapse during the last year or at least 2 relapses in the preceding 2 years, up to 4 points on the Expanded Disability Status Scale (EDSS), ability to provide the written informed consent, and an appropriate venous access for blood drawing. The most important exclusion criterion was any immunosuppression including interferon beta administered up to 6 months before the administration of the Tregs preparation. The only exception were glucocorticoids which could be administered as the treatment for relapses only. Other exclusion criteria included: other autoimmune diseases; diagnosed immunodeficiencies; presence or history of active infections, including hepatitis B, hepatitis C, HIV, tuberculosis (TB), systemic fungal infections; any history of malignancy; diagnosed cytopenias; elevated thrombotic activity or history of past thrombosis; hospitalization for cardiovascular events in the last 2 years before the inclusion; increased intracranial pressure defined as the papilledema; any retinopathy; arterial hypertension; presence or history of macroalbuminuria; excessive anxiety of the patient related to the procedures; any medical condition that, in the opinion of the investigator, may interfere with safe participation in the trial; known active alcohol or substance abuse; positive pregnancy test (for female subjects), unwillingness to use effective contraceptive measures during the study and for 4 months after discontinuation, when appropriate: intent to procreate during the study or within 4 months after discontinuation, when appropriate (for male subjects).

The follow-up started at administration of Tregs (day “0”) and lasted 12 months with the visits at: +14 days, +3 months, +6 months, +9 months, and +12 months post-administration. The endpoints measured included the amount and intensity of the therapy side effects, the number of annual relapses, worsening on the EDSS scale by at least 1 point, changes in the Multiple Sclerosis Functional Composite (MSFC) scale, changes in MRI according to the MAGNIMS 2015 consensus, changes in quality of Life Questionnaire (QOL), peripheral blood lymphocyte immunophenotype, and serum cytokines levels.

TABLE 1
Epidemiological characteristics of the patients

| | Intravenous (iv.) | Intrathecal (tc.) |
|---|---|---|
| Trait | administration | administration |
| Age (years)-median(min-max) | 28 (19-51) | 34 (26-45) |
| Sex (M/F) n | 6/5 | 3/0 |
| Age at diagnosis (years)- | 25 (18-39) | 31 (25-44) |
| median(min-max) | | |
| Disease duration at the | 5 (0.3-13) | 1.5 (1-3) |
| recruitment to the study | | |
| (years)-median(min-max) | | |

### Manufacturing and Administration of Tregs

The preparation of Tregs was manufactured under Good Manufacturing Practice (GMP) conditions similarly to our previous trials [10-13].

The cells were isolated from the patients' venous peripheral blood (450 ml) with HEPA-enclosed FACS sorter (Influx, BDBioscience, USA) using exchangeable sterile sample lines to the following phenotype CD3+CD4⁺CD25^{high}CD127⁻lin⁻doublet⁻. The sort itself was based on the staining and gating of the cells with GMP-grade monoclonal antibodies from Miltenyi Biotec, Germany (fluorochrome/class/clone): anti-CD4 (Vio-blue, IgG1, M-T466), anti-CD25 (PE, IgG1, 3G10) and anti-CD127 (APC, IgG1, MB15-18C9). An average post-sort Tregs purity was ≈98% (range 97-100%). The phenotype and impurities were additionally confirmed from post-sort sample of Tregs using monoclonal antibodies from BDBiosciences, Poland: (fluorochrome/class/clone): anti-CD3 (PacificBlue, IgG1, UCHT1), anti-CD4 (V-500, IgG1, RPA-T4), anti-CD8 (PerCP IgG1, SK1), anti-CD19 (PerCP, IgG1, 4G7), CD14 (PerCP, IgG2b, MpP9), anti-CD16 (PerCP-Cy5.5, IgG1, 3G8), anti-CD25 (PE, IgG1, M-A251), and anti-CD127 (APC, IgG1, hIL-7R-M21).

For intravenous administration, the expansion of Tregs was performed using clinical-grade anti-CD3/anti-CD28 beads (Miltenyi Biotec), interleukin 2 (aldesleukin, Novartis), and inactivated autologous serum for up to 14 days [median (min-max)=11(10-14)]. The medium (X-Vivo20, Lonza) was supplemented with 10% serum and 1000 UI/ml of IL2 throughout the entire expansion. The beads were added to the cells in the 1:1 ratio at the beginning of expansion and then during passages on days +7, +8 and +9 to restore 1:1 ratio. The culture was washed out from beads and left in 10% serum and low level of IL2 (100 UI/ml) for the last 24-48 h of the culture. The sentinel culture with autologous CD4+ Tconvs was performed in 10% serum and low level of IL2 (100 UI/ml) as a source of T responders for functional tests. The final product on release kept FoxP3 expression above 90% [median (min-max)=91%(90-97)]; CD62L expression above 80% [median (min-max)=87%(81-95)]; passed IFNγ suppression assay and microbiological tests were negative. The quality control of the cultures was performed on day +7 and on the release of the product. IFNγ suppression assay was performed as previously described [14]. Briefly, a sample of Tregs from the expansion cultures (washed out from the beads and left resting for at least 24 h) were cocultured with autologous sentinel Tconv cells in 1:1 ratio. The controls consisted of the cultures of Tconvs or Tregs only, either stimulated or not stimulated to produce IFNγ. Immediately prior to the assay, Tconvs were stained with cell tracer CFSE (CFDA kit Thermo, USA) in order to distinguish them from Tregs and therefore it was possible to give separately the proportions of IFNγ-positive Tregs and Tconvs at the end of the assay. The stimulation of the cultures and staining was performed with intracellular staining kit (BDBiosciences, Poland) according to the manufacturer description. The cultures were stimulated with 50 ng/ml of phorbol 12-myristate 13-acetate, 500 ng/ml of ionomycin (Sigma, Poland) and 2 μl/ml of cytokine leakage inhibitor GolgiPlug (BDBiosciences, Poland) for 5 h. Then, the cells were stained with anti-IFNγ antibodies. The positive readout of the assay was the suppression of IFNγ production by Tconvs cocultured with Tregs by at least 25% [median(min−max)=69% (52−95)], when compared to the production of IFNγ in the cultures with Tconvs only. The production of IFNγ by Tregs never exceeded 2% of the cells. The microbial safety was confirmed through negative results of microbiology cultures of supernatants from expansion media (BD Bactec system, BDBiosciences, Europe), negative endotoxin tests from supernatants of expansion media (Endosafe—PTS Endotoxin Cartridge/Cartridge reader, Charles River, USA), negative Gram staining of the supernatants from expansion media (Gram Stain Kit, BDBiosciences, Europe) and the absence of genetic material of HBV, HCV, HIV-1 and HIV-2 in the product (Cobas MPX, Roche, Europe). The patients were followed for any adverse symptoms related to the possible contamination of the product until all microbial post-release results were confirmed negative. The ready-to-use preparation of Tregs had to be administered within 2 hours of the release from the tissue establishment. The final dose was 40×10⁶ of Tregs/kg b.w. Upon release, the preparation was washed out completely, suspended in 250 ml of 0.9% NaCl for injection (Polfa, Warsaw), and then administered in slow intravenous infusion to the patient.

For patients treated intrathecally, 1 mln (1×10⁶) of freshly isolated Tregs (without expansion) was examined according to the release criteria described above and then suspended in 10 ml of 0.9% NaCl. Afterwards, it was administered in a slow injection during L4/L5 or L5/S1 lumbar puncture through a puncture needle. There was a 6-hour bed regimen post-injection.

### Clinical Assessment

Apart from routine physical/neurological examinations at the site visits, patients were also assessed according to the EDSS and MSFC scales by certified neurologists [15] to monitor the disease progression and according to EQ-5D questionnaire to monitor the quality of life [16]. The following lab tests were performed (only significantly abnormal values are shown): complete blood count, metabolic, kidney and liver panels, C-reactive protein levels, urinalysis.

### MRI Assessment

MRI of the brain was performed according to the MAGNIMS 2015 standard protocol (3D T1-weighted, 3D T2-FLAIR, 3D T2-weighted, and post-single-dose gadolinium-enhanced T1-weighted imaging, all with a nongapped section thickness of ≤3 mm, and a DWI sequence (5-mm section thickness, 1.5 Tesla Magnetom Aera, Siemens, Germany). MRI was performed during visits at +3 months, +6 months, and +12 months post-administration. The assessment of lesions and their progression were made using BrainMagix software (Brussels, Belgium) and Philips Intellispace Portal 10, the total number of plaques and contrast-enhanced plaques were counted by two observers.

### Immune Responses

Immune phenotyping was performed using ten-color panels to follow CD3⁺CD4⁺CD25^{high}CD127⁻FoxP3⁺ Tregs and CD3⁺CD4⁺CD25^{low/-}CD127⁺FoxP3⁻ Tconvs in the peripheral blood. In both populations, the expression of antigens important for the functioning of these subsets was followed. We specifically determined the percentage of naïve/memory subsets based on the following phenotypes: naïve/Tn (CD62L⁺CD45RA⁺), central memory/Tcm (CD62L⁺CD45RA⁻), and effector memory/Tem (CD62L⁻CD45RA⁻). CD3⁺CD4⁺CD25^{high}CD127⁻FoxP3⁺ Tregs were further divided based on the expression of transcription factor Helios into the peripheral [pTreg Helios(−)] and thymic [tTreg Helios(+)] subsets [17](FIG. 4).

The following anti-human monoclonal antibodies purchased from BDBiosciences, Poland, were used in this procedure (fluorochrome/class/clone): anti-CD3 (PacificBlue/IgG1/UCHT1 or V500-C/IgG1/clone SK7), anti-CD4 (PerCP or AlexaFluor700/IgG1/RPA-T4), anti-CD25 (PE or BV786/IgG1/M-A251), anti-CD127 (FITC or BUV737/IgG1/hIL-7R-M21), anti-CD45RA (PE-Cy7/IgG1/L48), anti-CD73 (BUV737/IgG1/AD2), anti-CD279 (BV605/IgG1/EH12.1), anti-CD137 (BV650/IgG1/4B4-1), anti-CD134 (BV711/IgG1/ACT35), anti-CD152 (BV786/IgG1/BNI3) anti-CD18 (FITC/IgG1/L130), anti-CD184 (PE-CF594/IgG1/12G-5), anti-CD194 (BV605/IgG1/1G1), anti-CD39 (BV650/IgG1/TU66 or BUV737/IgG1/TU66), and anti-CD103 (BUV395/IgG1/Ber-ACT8). Anti-CD62L (APC-Cy7/IgG1/3B5) was supplied by Invitrogen, USA; the FoxP3 staining kit and anti-Helios (eFluor450/IgG1/22F6), by ebioscience/thermoFisher, USA; anti-CCR8 (PerCP/IgG1/91704) and anti-CCR10 (PE/IgG1/314305), by R&D/biotechne, UK.

Serum levels of 38 cytokines: IFNalpha2, IFNgamma, IL10, IL12p40, IL12p70, IL13, IL15, sCD40L, IL17, IL2, IL1RA, IL1alpha, IL1beta, IL3, IL4, IL5, IL6, IL9, TNFalpha, TNFbeta, EGF, FGF-2, TGF-alpha, G-CSF, GM-CSF, VEGF, FLT-3L, IL7, Eotaxin, CX3CL-1, CXCL-1, MCP-3, CCL22, IL8, IP-10, MCP-1, MIP-1alpha, and MIP-1beta were measured with the Bead based Multiplex Assay on luminex analyzer (Merck, USA). All assays were performed according to the manufacturers' instructions.

### Statistical Analysis

Data were computed with the software Statistica 12.0 (Statsoft, Poland). Cluster analysis was performed with ClustVis software (https://biit.cs.ut.ee/clustvis/#mathematics). The analysis was carried out with nonparametric tests. P≤0.05 was considered statistically significant.

### Results

### 1.1. Safety

No serious adverse events were reported throughout the trial. Moderate adverse effects were noted in patients treated with Tregs intravenously (iv.). The most common adverse effects were relapses and progression of lesions in the CNS. Interestingly, no adverse effects were noted in patients administered with Tregs intrathecally (tc.) (Table 2).

TABLE 2
Adverse effects in the trial

| | | |
|---|---|---|
| Adverse event | Number of patients/events | Severity |

Tregs administered intravenously

| | | |
|---|---|---|
| Relapse of MS | 5/12 (3 patients experienced 3 relapses, | Moderate |
| | 1 patient experienced 2 relapses, | |
| | and 1 patient experienced 1 relapse) | |
| Progression of changes | 5/5 | Moderate |
| in the CNS on MRI | | |
| Progression of visual impairment | 1/1 | Moderate |
| Liver injury (increased ASPAT | 1/1 | Moderate |
| and ALAT without clinical | | |
| symptoms, unknown etiology) | | |

Tregs administered intrathecally
No adverse events reported

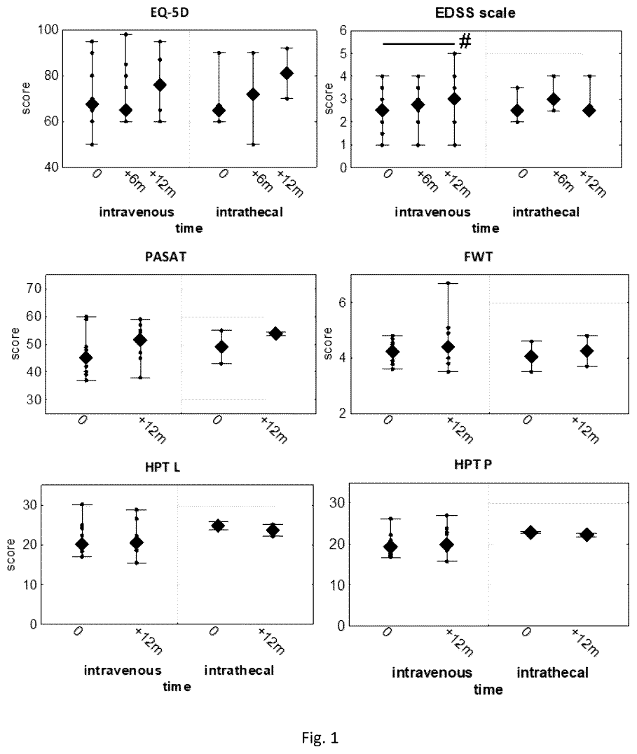

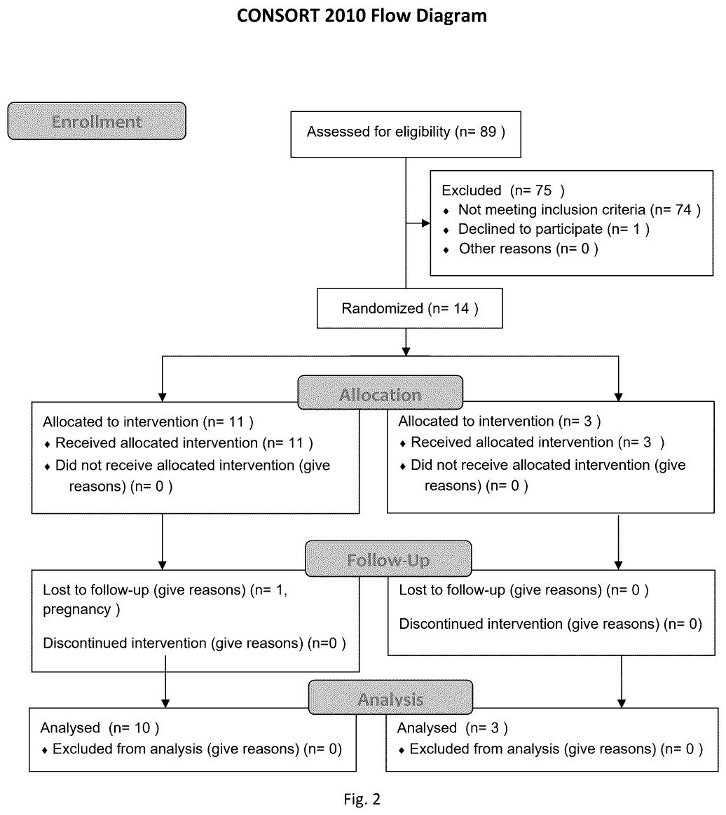

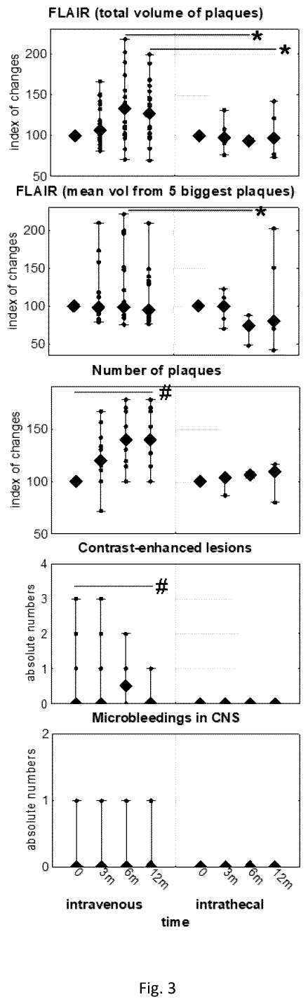

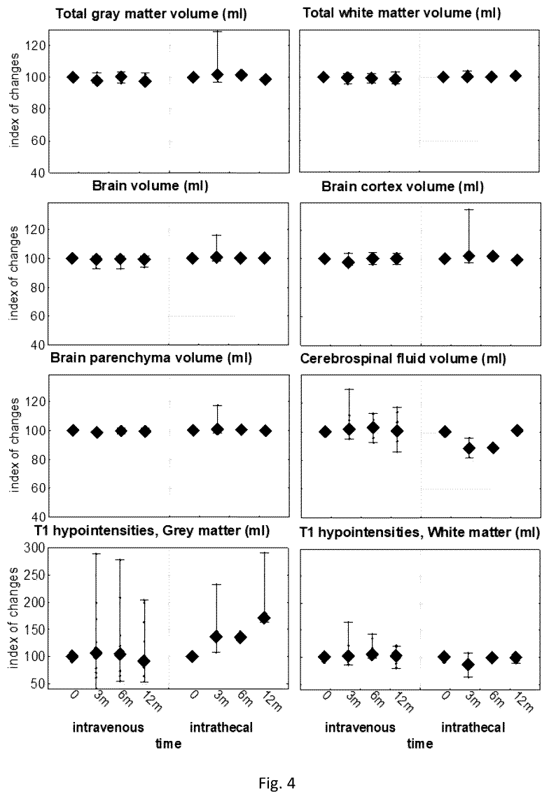

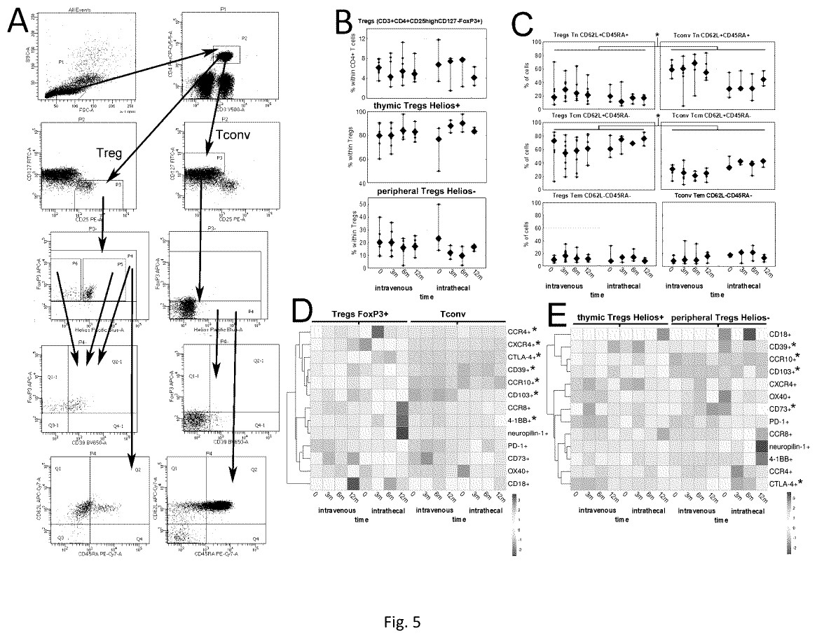

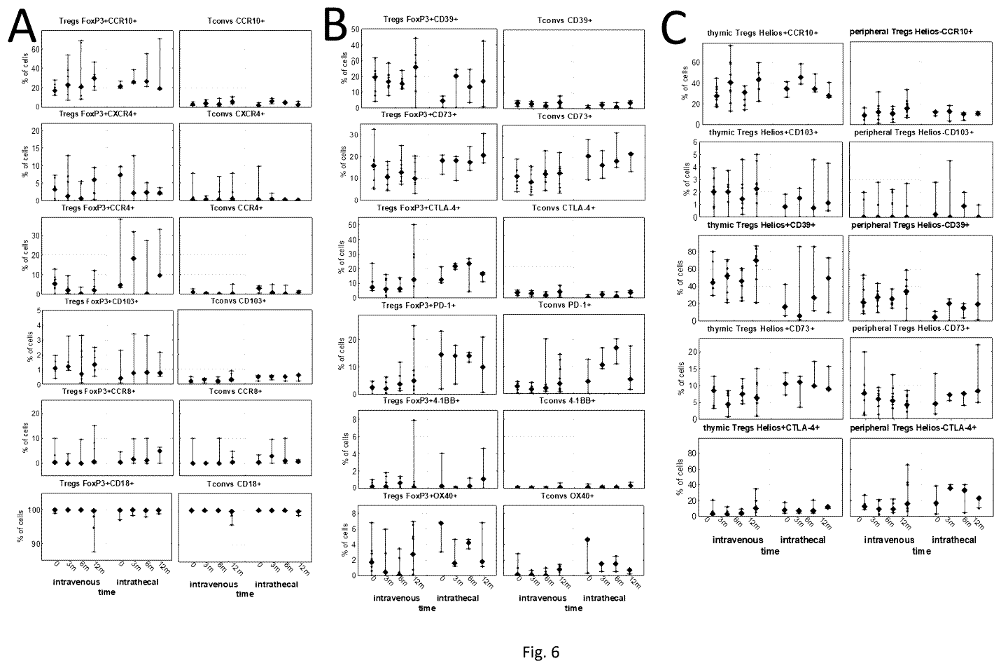

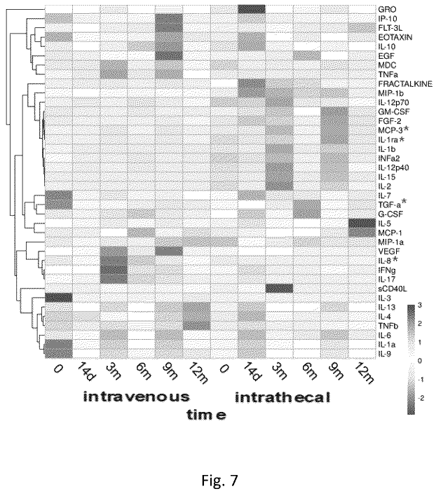

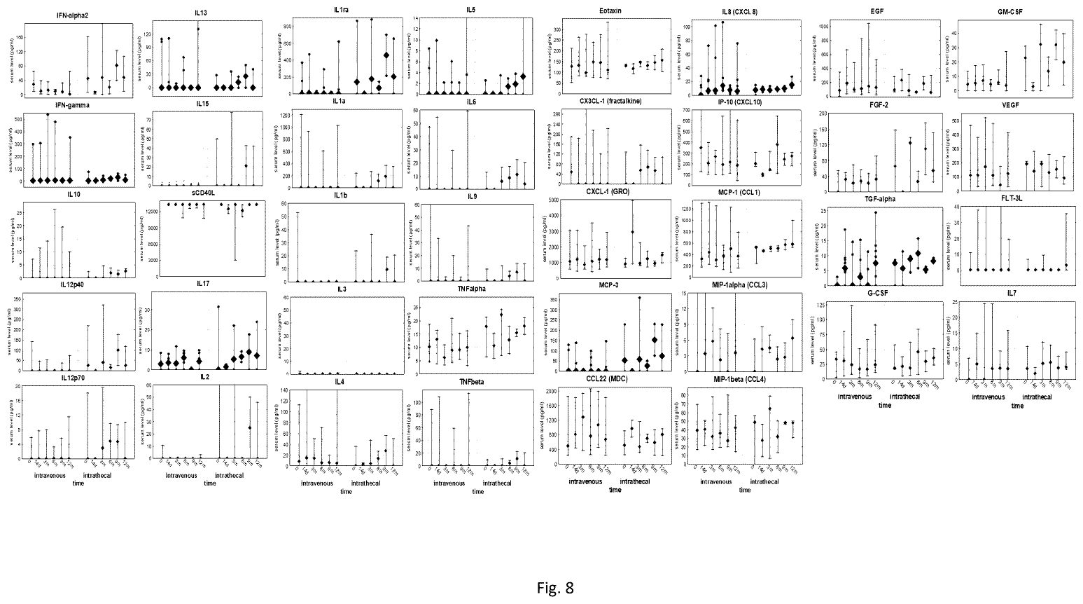

The analysis of the quality of life revealed no deterioration in the self-assessment using EQ-5D form. The results were similar in both groups throughout the follow-up (all tests p>0.05, FIG. 1).

### 1.2. Efficacy—Clinical

The clinical status assessed using EDSS scale did not differ between the groups throughout the study [Kruskal-Wallis ANOVA: day 0: H=0.18 p=0.66; 6m: H=0.36 p=0.54; 12m: H=0.029 p=0.86](FIG. 1). However, one-year deterioration on the EDSS scale within the tc. group and within the iv. group was from 0 to 0.3 and from 0 to 1, respectively. In the iv. group, 3 out of 10 subjects revealed a deterioration higher than 1 point on the EDSS scale. No such a deterioration was seen in those treated intrathecally. Total of 12 relapses were noted in 5 patients treated intravenously with the frequency from 1 to 3 episodes per year during the follow-up. At the same time, no relapses were observed in the tc. group.

The clinical status assessed using the MSFC scale did not change in any group and did not differ between the groups in any of the scale components throughout the study (all tests p>0.05, FIG. 1).

### 1.3. Efficacy—MRI

When compared to iv. group, the analysis of MRI scans revealed a lower activity of the disease in the tc. group (FIG. 3).

The FLAIR sequence revealed that the total volume of plaques in the CNS throughout the follow-up increased in iv. group while it did not change in tc. group [Friedman's ANOVA: iv.: χ²=12.79 p=0.005; tc.: χ²=4.5 p=0.21]. The difference between the groups was significant at 6 and 12 month of the follow up [Kruskal-Wallis ANOVA: 3m: H=1.65 p=0.19; 6m: H=6.14 p=0.013; 12m: H=5.33 p=0.047]. The difference was also seen when the volume of the five biggest plaques [Kruskal-Wallis ANOVA: 3m: H=0.01 p=0.91; 6m: H=7.77 p=0.005; 12m: H=2.34 p=0.067] and the number of new plaques [Kruskal-Wallis ANOVA: 3m: H=3.76 p=0.15; 6m: H=5.10 p=0.076; 12m: H=4.61 p=0.091] were compared between the groups. Interestingly, it was the increasing number of the plaques in iv. group [Friedman's ANOVA for the number of the plaques: iv.: χ²=20.77 p=0.0001; tc.: χ²=5.5 p=0.13] rather than the changes of the existing the biggest plaques [Friedman's ANOVA for mean volume from 5 biggest plaques: iv.: χ²=3.66 p=0.30; tc.: χ²=3.90 p=0.27] were responsible for the increase in the total volume of the plaques during the follow-up. In addition, contrast-enhanced T1 lesions in iv. group decreased significantly at the end of the trial. This was not the case of tc. patients as these lesions were not seen in this group throughout the follow-up [Friedman's ANOVA: iv.: χ²=11.41 p=0.009; tc.: all numbers ‘0’]. Neither the volume of the main CNS structures or the volume of T1 hypointensiveness differed between the groups (FIG. 6).

### 1.4. Immune Response

### 1.4.1. Treg Subsets

There were no significant changes in the level of FoxP3+ Tregs and Tconvs throughout the follow-up or between the groups (all tests p>0.05, FIG. 5). However, Tregs differed from Tconvs in several measured subsets in all patients, regardless the route of administration of Tregs. When all patients were taken into account, when compared Tregs to Tconvs, Tregs contained mostly Tcm phenotype (50% or more), while Tconvs contained mostly Tn phenotype (50% or more) (FIG. 5C and Table 1S). We have also found that Tregs expressed several receptors, such as chemokine receptors CCR10, CXCR4, CCR4, integrin CD103, ectonucleotidase CD39, and two costimulatory molecules—CTLA-4 and 4-1BB, which were almost undetectable on Tconvs (FIG. 6A,B and Table 1S). The difference between Tregs and Tonvs in the expression of these receptors was confirmed with cluster analysis (FIG. 5D).

In addition, around 20% of Tregs in all patients did not express the transcription factor Helios suggesting peripheral origin of these cells (FIG. 5B). Having that in mind, we performed deeper analysis dividing Tregs into thymic FoxP3+Helios(+) tTregs and peripheral FoxP3+Helios(−) pTregs. When compared, tTregs contained higher percentage of CCR10⁺ cells, CD103⁺ cells, CD73⁺ cells, and CD39⁺ cells, while pTreg contained higher percentage of CTLA-4⁺ cells (FIG. 6-C and Table 1S). The cluster analysis confirmed that the higher expression of CCR10, CD103, CD39 and lower expression of CTLA-4 receptors differs tTregs from pTregs (FIG. 5E).

TABLE 1S
The significance of differences between CD4⁺ T cell subsets of Tregs and Tconvs
(Tregs vs. Tconvs) as well as thymic and peripheral subsets of Tregs
(tTregs vs. pTregs) in all patients, regardless the route of administration
(Kruskal-Wallis ANOVA, p ≤ 0.05 as significant in bold)

| | | Tregs vs. Tconvs | | tTregs vs. pTregs | |
|---|---|---|---|---|---|
| Phenotype | time | H | p | H | p |
| Tn CD45RA⁺CD62L⁺ | 0 | 6.86 | 0.008 | 0.21 | 0.64 |
| | 3 m | 6.70 | 0.009 | 1.71 | 0.19 |
| | 6 m | 6.84 | 0.008 | 0.09 | 0.75 |
| | 12 m | 2.28 | 0.13 | 0.34 | 0.55 |
| Tcm CD45RA⁻CD62L⁺ | 0 | 9.76 | 0.002 | 2.70 | 0.09 |
| | 3 m | 6.18 | 0.012 | 0.21 | 0.64 |
| | 6 m | 8.69 | 0.003 | 3.32 | 0.08 |
| | 12 m | 15.39 | 3.1 × 10−4 | 0.066 | 0.93 |
| Tem CD45RA⁻CD62L⁻ | 0 | 0.25 | 0.61 | 0.09 | 0.95 |
| | 3 m | 0.14 | 0.70 | 0.35 | 0.76 |
| | 6 m | 0.16 | 0.89 | 1.07 | 0.29 |
| | 12 m | 3.50 | 0.06 | 0.64 | 0.56 |
| CCR10⁺ | 0 | 20.28 | 4.7 × 10−6 | 17.49 | 5.7 × 10−5 |
| | 3 m | 16.34 | 6.7 × 10−5 | 15.39 | 3.2 × 10−4 |
| | 6 m | 16.34 | 8.2 × 10−5 | 14.52 | 5.3 × 10−4 |
| | 12 m | 18.78 | 7.4 × 10−6 | 15.20 | 1.7 × 10−4 |
| CXCR4⁺ | 0 | 3.52 | 0.046 | 0.49 | 0.48 |
| | 3 m | 3.57 | 0.042 | 3.58 | 0.085 |
| | 6 m | 3.04 | 0.048 | 0.084 | 0.77 |
| | 12 m | 8.70 | 0.033 | 0.23 | 0.62 |
| CCR4⁺ | 0 | 10.23 | 0.001 | 0.61 | 0.43 |
| | 3 m | 7.58 | 0.006 | 0.27 | 0.59 |
| | 6 m | 2.39 | 0.091 | 0.15 | 0.69 |
| | 12 m | 5.24 | 0.022 | 1.35 | 0.24 |
| CD103⁺ | 0 | 12.67 | 4.4 × 10−4 | 2.45 | 0.11 |
| | 3 m | 12.62 | 4.4 × 10−4 | 3.43 | 0.048 |
| | 6 m | 5.92 | 0.014 | 0.86 | 0.35 |
| | 12 m | 12.79 | 3.9 × 10−4 | 10.22 | 0.001 |
| CCR8⁺ | 0 | 0.19 | 0.66 | 0.41 | 0.51 |
| | 3 m | 0.01 | 0.91 | 0.26 | 0.60 |
| | 6 m | 0.003 | 0.94 | 0.62 | 0.42 |
| | 12 m | 1.01 | 0.31 | 1.14 | 0.28 |
| CD18⁺ | 0 | 1.39 | 0.12 | 1.29 | 0.13 |
| | 3 m | 0.68 | 0.41 | 0.01 | 0.91 |
| | 6 m | 1.69 | 0.19 | 1.91 | 0.16 |
| | 12 m | 0.02 | 0.87 | 0.15 | 0.68 |
| CD39⁺ | 0 | 13.85 | 2.2 × 10−4 | 5.93 | 0.014 |
| | 3 m | 13.45 | 2.8 × 10−4 | 4.42 | 0.035 |
| | 6 m | 8.01 | 0.004 | 4.08 | 0.043 |
| | 12 m | 5.44 | 0.019 | 6.18 | 0.012 |
| CD73⁺ | 0 | 0.72 | 0.39 | 2.96 | 0.06 |
| | 3 m | 0.23 | 0.62 | 1.07 | 0.31 |
| | 6 m | 0.28 | 0.59 | 6.18 | 0.013 |
| | 12 m | 0.07 | 0.78 | 3.37 | 0.052 |
| CTLA-4⁺ | 0 | 9.06 | 0.002 | 3.05 | 0.048 |
| | 3 m | 13.07 | 3.1 × 10−4 | 6.97 | 0.008 |
| | 6 m | 14.10 | 2.5 × 10−4 | 5.07 | 0.023 |
| | 12 m | 12.53 | 4.5 × 10−4 | 4.20 | 0.042 |
| PD-1⁺ | 0 | 0.21 | 0.64 | 0.008 | 0.92 |
| | 3 m | 0.16 | 0.68 | 0.19 | 0.62 |
| | 6 m | 0.12 | 0.72 | 0.40 | 0.52 |
| | 12 m | 0.63 | 0.42 | 0.31 | 0.57 |
| 4-1BB⁺ | 0 | 4.16 | 0.041 | 3.58 | 0.06 |
| | 3 m | 2.97 | 0.071 | 0.08 | 0.76 |
| | 6 m | 6.22 | 0.016 | 1.78 | 0.18 |
| | 12 m | 1.24 | 0.26 | 0.79 | 0.37 |
| OX40⁺ | 0 | 1.83 | 0.09 | 2.18 | 0.13 |
| | 3 m | 1.46 | 0.11 | 0.06 | 0.75 |
| | 6 m | 1.61 | 0.19 | 0.25 | 0.61 |
| | 12 m | 4.76 | 0.021 | 0.24 | 0.62 |

### 1.4.2. Cytokines

The study included also the array of 38 different cytokines measured in the sera of patients. When compared to the intravenously treated patients, those treated intrathecally revealed higher levels of some factors associated with inflammation, such as MCP-3, IL1RA and IL8. Interestingly, also the level of brain trophic factor TGFα was higher in tc. group than in the iv. group (Table 2S, FIG. 8). The levels of MCP-3, IL1RA positioned in the same cluster differing tc. group from iv. group (FIG. 7). The levels of other measured cytokines did not differ between the trial groups or within each group throughout the follow-up.

TABLE 2S
The significance of differences in the serum level of cytokines
between iv. patients and tc. patients. Only the statistics
of cytokines with significant differences is presented.
(Kruskal-Wallis ANOVA, p ≤ 0.05 as significant in bold)

| | | iv. patients vs. tc. patients | |
|---|---|---|---|
| cytokine | time | H | p |
| MCP-3 | 0 | 2.74 | 0.048 |
| | 14 days | 0.48 | 0.48 |
| | 3 m | 3.80 | 0.03 |
| | 6 m | 0.02 | 0.88 |
| | 9 m | 5.83 | 0.01 |
| | 12 m | 2.37 | 0.048 |
| IL8 (CXCL8) | 0 | 0.15 | 0.69 |
| | 14 days | 0.22 | 0.63 |
| | 3 m | 0.20 | 0.65 |
| | 6 m | 2.89 | 0.041 |
| | 9 m | 2.15 | 0.069 |
| | 12 m | 2.92 | 0.046 |
| IL1RA | 0 | 0.97 | 0.32 |
| | 14 days | 3.21 | 0.037 |
| | 3 m | 2.14 | 0.08 |
| | 6 m | 0.07 | 0.77 |
| | 9 m | 4.11 | 0.024 |
| | 12 m | 2.65 | 0.047 |
| TGF-α | 0 | 4.25 | 0.03 |
| | 14 days | 0.94 | 0.54 |
| | 3 m | 2.21 | 0.08 |
| | 6 m | 3.50 | 0.03 |
| | 9 m | 3.05 | 0.04 |
| | 12 m | 0.97 | 0.37 |

### REFERENCES

1. Dendrou C A, Fugger L, Friese M A (2015) Immunopathology of multiple sclerosis. Nature Reviews Immunology 15:545
2. Abbas A K, Benoist C, Bluestone J A, Campbell D J, Ghosh S, Hori S, Jiang S, Kuchroo V K, Mathis D, Roncarolo M G, Rudensky A, Sakaguchi S, Shevach E M, Vignali D A, Ziegler S F (2013) Regulatory T cells: recommendations to simplify the nomenclature. Nat Immunol 14:307-308
3. Kleinewietfeld M, Hafler D A (2014) Regulatory T cells in autoimmune neuroinflammation. Immunological Reviews 259:231-244
4. Sakaguchi S, Miyara M, Costantino C M, Hafler D A (2010) FOXP3+ regulatory T cells in the human immune system. Nat Rev Immunol 10:490-500
5. Miyara M, Gorochov G, Ehrenstein M, Musset L, Sakaguchi S, Amoura Z (2011) Human FoxP3+ regulatory T cells in systemic autoimmune diseases. Autoimmun Rev 10:744-755
6. Trzonkowski P, Szmit E, Mysliwska J, Mysliwski A (2009) Ex vivo expansion of CD4(+)CD25(+) T regulatory cells for immunosuppressive therapy. Cytometry A 75:175-188
7. Campbell D J (2015) Control of Regulatory T Cell Migration, Function, and Homeostasis. The Journal of Immunology 195:2507-2513
8. Gliwinski M, Iwaszkiewicz-Grześ D, Trzonkowski P (2017) Cell-Based Therapies with T Regulatory Cells. BioDrugs 31:335-347
9. Trzonkowski P, Bacchetta R, Battaglia M, Berglund D, Bohnenkamp H R, ten Brinke A, Bushell A, Cools N, Geissler E K, Gregori S, Marieke van Ham S, Hilkens C, Hutchinson J A, Lombardi G, Madrigal J A, Marek-Trzonkowska N, Martinez-Caceres E M, Roncarolo M G, Sanchez-Ramon S, Saudemont A, Sawitzki B (2015) Hurdles in therapy with regulatory T cells. Sci Transl Med 7:304ps318
10. Trzonkowski P, Bieniaszewska M, Juścìńska J, Dobyszuk A, Krzystyniak A, Marek N, Mysliwska J, Hellmann A (2009) First-in-man clinical results of the treatment of patients with graft versus host disease with human ex vivo expanded CD4+CD25+CD127− T regulatory cells. Clin Immunol 133:22-26
11. Marek-Trzonkowska N, Myśliwiec M, Dobyszuk A, Grabowska M, Techmanska I, Juscinska J, Wujtewicz M A, Witkowski P, Mlynarski W, Balcerska A, Mysliwska J, Trzonkowski P (2012) Administration of CD4+CD25highCD127− Regulatory T Cells Preserves β-Cell Function in Type 1 Diabetes in Children. Diabetes Care 35:1817-1820
12. Marek-Trzonkowska N, Myśliwiec M, Iwaszkiewicz-Grzes D, Gliwiński M, Derkowska I, \grave{Z}alińska M, Zieliński M, Grabowska M, Zielińska H, Piekarska K, Jaźwińska-Curyłło A, Owczuk R, Szadkowska A, Wyka K, Witkowski P, Młynarski W, Jarosz-Chobot P, Bossowski A, Siebert J, Trzonkowski P (2016) Factors affecting long-term efficacy of T regulatory cell-based therapy in type 1 diabetes. Journal of Translational Medicine 14:332
13. Marek-Trzonkowska N, Myśliwiec M, Dobyszuk A, Grabowska M, Derkowska I, Juścinska J, Owczuk R, Szadkowska A, Witkowski P, Młynarski W, Jarosz-Chobot P, Bossowski A, Siebert J, Trzonkowski P (2014) Therapy of type 1 diabetes with CD4+CD25highCD127− regulatory T cells prolongs survival of pancreatic islets—Results of one year follow-up. Clinical Immunology 153:23-30
14. Marek N, Bieniaszewska M, Krzystyniak A, Juscinska J, Mysliwska J, Witkowski P, Hellmann A, Trzonkowski P (2011) The time is crucial for ex vivo expansion of T regulatory cells for therapy. Cell Transplant 11-12:1747-1758
15. http://www.nationalmssociety.org/for-professionals/researchers/clinical-study-measures/msfc/index.aspx [online]
16. EuroQol Group (1990) EuroQol—a new facility for the measurement of health-related quality of life. Health Policy 16:199-208
17. Thornton A M, Korty P E, Tran D Q, Wohlfert E A, Murray P E, Belkaid Y, Shevach E M (2010) Expression of Helios, an Ikaros Transcription Factor Family Member, Differentiates Thymic-Derived from Peripherally Induced Foxp3+ T Regulatory Cells. The Journal of Immunology 184:3433-3438
18. International Multiple Sclerosis Genetics Consortium; Wellcome Trust Case Control Consortium (2011) Genetic risk and a primary role for cell-mediated immune mechanisms in multiple sclerosis. Nature 476:214-219
19. Raddassi K, Kent S C, Yang J, Bourcier K, Bradshaw E M, Seyfert-Margolis V, Nepom G T, Kwok W W, Hafler D A (2011) Increased Frequencies of Myelin Oligodendrocyte Glycoprotein/MHC Class II-Binding CD4 Cells in Patients with Multiple Sclerosis. The Journal of Immunology 187:1039
20. Nylander A, Hafler D A (2012) Multiple sclerosis. The Journal of Clinical Investigation 122:1180-1188
21. Zhang X, Koldzic DN, Izikson L, Reddy J, Nazareno R F, Sakaguchi S, Kuchroo V K, Weiner H L (2004) IL-10 is involved in the suppression of experimental autoimmune encephalomyelitis by CD25+CD4+ regulatory T cells. International Immunology 16:249-256
22. Haas J, Hug A, Viehover A, Fritzsching B, Falk C S, Filser A, Vetter T, Milkova L, Korporal M, Fritz B, Storch-Hagenlocher B, Krammer P H, Suri-Payer E, Wildemann B (2005) Reduced suppressive effect of CD4+CD25high regulatory T cells on the T cell immune response against myelin oligodendrocyte glycoprotein in patients with multiple sclerosis. European Journal of Immunology 35:3343-3352
23. Venken K, Hellings N, Hensen K, Rummens J L, Medaer R, D'hooghe M B, Dubois B, Raus J, Stinissen P. (2006) Secondary progressive in contrast to relapsing-remitting multiple sclerosis patients show a normal CD4+CD25+ regulatory T-cell function and FOXP3 expression. Journal of Neuroscience Research 83:1432-1446
24. Feger U, Luther C, Poeschel S, Melms A, Tolosa E, Wiendl H (2007) Increased frequency of CD4+CD25+ regulatory T cells in the cerebrospinal fluid but not in the blood of multiple sclerosis patients. Clinical & Experimental Immunology 147:412-418
25. Lee M J, Bing S J, Choi J, Jang M, Lee G, Lee H, Chang B S, Jee Y, Lee S J, Cho I H (2016) IKKβ-mediated inflammatory myeloid cell activation exacerbates experimental autoimmune encephalomyelitis by potentiating Th1/Th17 cell activation and compromising blood brain barrier. Molecular Neurodegeneration 11:54
26. Hoffmann P E R, Boeld T J, Doser K, Piseshka B, Andreesen R, Edinger M (2006) Only the CD45RA+ subpopulation of CD4+CD25high T cells gives rise to homogeneous regulatory T-cell lines upon in vitro expansion. Blood 108:4260-4267
27. Shevach E M, Thornton A M (2014) tTregs, pTregs, and iTregs: similarities and differences. Immunological Reviews 259:88-102
28. Edinger M H P (2009) Regulatory T cells for the prevention of graft-versus-host disease: professionals defeat amateurs. Eur J Immunol 39:2966-2968

1. A method for treating multiple sclerosis in a subject, said method comprising intrathecally administering a population of CD3+CD4+CD25+CD127− T regulatory cells to the subject.

2. The method of claim 1, wherein multiple sclerosis is relapsing-remitting multiple sclerosis.

3. The method of claim 1, wherein intrathecal administration is via injection through L4/L5 or L5/S1 lumbar puncture.

4. The method of claim 1, wherein the population of cells is formulated in 0.9% NaCl.
